(19) United States
(12) Patent Application Publication
(10) Pub. No.: US 2016/0296582 A1
(43) Pub. Date: Oct. 13, 2016

(54) Mixture of Specific Essential Oils for the treatment of arthritis

(71) Applicant: Rahul Kapadia, Mount Waverley (AU)

(72) Inventor: Rahul Kapadia, Mount Waverley (AU)

(21) Appl. No.: 14/684,335

(22) Filed: Apr. 11, 2015

(51) Int. Cl. A61K 36/752; A61K 9/00; A61K 36/534

(52) U.S. Cl. CPC A61K 36/752; A61K 36/534; A61K 9/0014; A61K 9/0019

(57) ABSTRACT

The presept invention provides methods for the treatment of arthritis using a mixture of specific essential oils in a certain formula as a topical treatment. The essential oils used are Orange Oil (Citrus Sinensis) and Peppermint Oil (Mentha Piperita). These oils are categorised as “Active” and can be mixed with any “Non-Active” or “Carrier” oils, to apply topically to the effected area.

### BACKGROUND

Arthritis is a major disease effecting the bone or joint. This consists of inflammation, loss of bone, inflamed joints, wear and tear and eventual loss of cartilege or tissue. As a result the human body suffers pain and stiffness and loss of motion.

Arthritis usually affects the elderly as a result of continuous wear and tear.

The most common affected areas in the human body are the toes, ankle and joints within the foot, knees, hips, spine,back, neck, shoulder, elbow, wrist, fingers and joints within the hand.

The usual treatment of arthritis has ben through oral or injectible solutions, which sometimes can give rise to side-effects related to gastro intestinal or cardio vascular type issues.

The search for an all natural herbal treatment to be applied topically which provides relief from arthritic conditions is very important The present invention fulfills this issue.

U.S. Patents have been issued for herbal medicines for the treatment of various health related problems affecting the human and animal race. For example, U.S. Pat. No. 5,595,743 is for the preparation of herbal medicines by using a multi enzyme system and discloses various herbal compositions which include licorice extract (Glycyrrhiza) and Siegesbeckia, Sophora, Stemona and Tetrandra herbs.

U.S. Pat. No. 5,683,697 has been issued where the composition includes plant parts from the species Melia, Angepica, Dendrobium, Impatiens, Citrus Medica, Loranthus, Celosia, Cynanchum and Glehnia and a herbal formulation comprising extracts of the roots, rhizomes, and/or vegetation of Alphinia, Smilax, Tinospora, Tribulus, Withania and Zingiber. U.S. Pat. No. 6,194,469 discloses a method using cherry bioflavonoids.

### DISCLOSURE OF THE INVENTION

The present invention relates to a method for the treatment of arthritis or arthritic conditions.

In one embodiment, the invention relates to a method of treating, preventing, inhibiting or curing arthritis in a subject, which comprises administering to the subject a therapeutically effective compound consisting of the essential oil of citrus sinensis or otherwise known as ‘orange peel extract’. This oil is categorised as an ‘Active oil’.

In another embodiment, this is enriched with mentha piperita or otherwise known as ‘peppermint oil’. This oil is categorised as an ‘Active oil’.

In another embodiment, the ‘Non-Active oils’ or otherwise known as carrier oils, consist of any vegetable based oil, such as Coconut oil or otherwise known as cocos nucifera, Olive Oil or otherwise known as Olea Europaea, Canola Oil, Corn Oil, Cotton seed Oil, Flaxseed Oil, Linseed Oil, Palm Oil, Peanut Oil, Safflower Oil, Soybean Oil or Sunflower Oil.

In some embodiments, the route of administration is the topical application of the composition on the epidermis covering the areas of the joints.

In other embodiments, the route of administration can be injected into the affected area.

In some embodiments, the subject is a human.

In other embodiments the subject is an animal.

Orange oil is derived through an extraction method called cold-pressing. It yields approximately 0.3% -0.5%. It is known to have therapeutic properties amongst others of being, anti-inflammatory, anti-septic, diuretic, sedative, and carminative.

Peppermint oil is extracted by steam distillation with a yield of approximately 0.1%-1.0%. Its therapeutic properties include being an anti-septic, analgesic, anti-galactagogue, anti-phlogistic and anti-spasmodic.

### THE INVENTION

### Definitions

“Arthritis” is often referred to as a single disease. In fact, it is an umbrella term for more than 100 medical conditions that affect the musculoskeletal system, specifically joints where two or more bones meet. Arthritis-related problems include pain, stiffness, inflammation and damage to joint cartilage (the tissue that covers the ends of bones, enabling them to move against each another) and surrounding structures. This can result in joint weakness, instability and deformities that can interfere with the most basic daily tasks such as walking, driving a car and preparing food. While there are about 100 forms of arthritis, the three most significant are osteoarthritis, rheumatoid arthritis and gout. The most common forms of arthritis are:
Osteoarthritis
Rheumatoid arthritis
Gout
Ankylosing spondylitis
Juvenile arthritis
Systemic lupus erythematosus (lupus)
Scleroderma

“Mixture” refers to a combination of active components or ingredients together with the non-active components that are administered together.

“Subject” includes, but is not limited to, humans and animals

“Topical” refers to the mixture by applying, massaging, dabbing, spreading, spraying, swabbing or sponging on the epidermis of the affected area.

The above-mentioned compositions and methods of administration are meant to describe but not limit the methods and compositions of the present invention.

### Testing

In a Research Study published by the National Product Research in 2010, that the various varieties of ROC Red Orange Complex stated that ROC exerts anti-inflammatory effects through the suppression of COX-2 and iNOS expression.

The Journal of Chemical and Pharmaceutical Research 2013 studied and evaluated the antioxidant, anti-inflammatory and antibacterial properties of ethanolic extracts of Citrus sinensis peels and leaves. Results clearly indicate that free radical scavenging, antibacterial and anti-inflammatory properties of ethanolic extracts of Citrus sinensis are comparable to ascorbic acid, ciprofloxacin and aspirin used as standards respectively.

In a Research Article published by the Journal of Chemical & Pharmaceutical Research 2013, an Evaluation of anti-inflammatory, antibacterial and antioxidant properties of ethanolic extracts of Citrus sinensis peel and leaves. The conclusion was that sweet orange peels which are considered as waste materials of the fruit, and leaves could serve as potential antibacterial, anti-inflammatory and antioxidant agents.

The German Commission E Monographs state that Peppermint Oil has external use for Myalgia and Neuralgia.

### EXAMPLES

A middle aged man with severe arthritis afflicted on his left hand. As a result he is unable to have the full movement of his fingers and is unable to clench his fist. Within a short span of applying this mixture, he is able to gain better movement with his fingers and eventually able to clench his fist

A 51 year old man with osteoarthritis in his left foot, aggravated as a result of a recent fall. As a result of the event, the left foot collapsed ensuring that he was completely flat footed. Custom made foot arches were organised to assist the foot and to prevent the joints from touching each others, as he was suffering from pain and therefore lack of movement. On applying this mixture, the pain was removed and movement improved to the extent where he no longer needs the custom made arches.

A person suffering from arthritis of the knee, would often complain of inflammation and pain in his right knee after playing tennis. He had undertaken a few clean outs of the knee, but kept on having the same symptoms. On applying this mixture, not only was the inflammation and pain contained, but within a few months seemed to have cured his condition.

A 73 year old man was suffering with severe arthritis in his toe and therefore felt distinctly uncomfortable in wearing his shoes. 10 days into applying this mixture, he could wear his shoes and walk much more comfortably.

A middle aged lady with arthritic conditions in her knee could not walk mid size distances of up to a kilometre. On applying this mixture, was able to walk to her supermarket and back without any pain.

PATENT CITATIONS

| | Filing | Publication | | |
|---|---|---|---|---|
| Cited Patent | Date | Date | Applicant | Title |
| U.S. Pat. No. 6,444,238B1 | 10 Mar | 3 Sep 2002 | Richard | Using |
| | 2000 | | Weise | Mixture of |
| | | | | Plant |
| | | | | Extracts |
| WO2007139887A2 | 22 May | 6 Dec 2007 | Cargill Inc. | Methods for |
| | 2007 | | Sekhar | treating |
| | | | Boddupalli, | bone of joint |
| | | | Khalid | inflammation |
| | | | Mahmood, | |
| | | | David N | |
| | | | Liebotitz | |
| US20140363420A1 | 5 Jun | 11 Dec 2014 | Sweet Relief | Method and |
| | 2014 | | Inc. | Composition |
| | | | | for topical |
| | | | | pain relief |

NON PATENT CITATIONS

| | | | | |
|---|---|---|---|---|
| 1 | Parle | Orange: Range | International | IRJP |
| | Milind & | of Benefits | Research | 2012, 3(7) |
| | Chaturde | | Journal of | |
| | vi Dev | | Pharmacy | |
| | | | Department | |
| | | | of Food | |
| | | | Science, | |
| 2 | Robert T | Anti- | Rutgers | 28 Mar. |
| | Rosen & | inflammatory | University | 2004 |
| | Chi-Tang | activity of | | |
| | Ho | polymethoxyflavones | | |
| | | in sweet | | |
| | | orange (Citrus | | |
| | | sinensis) peel | | |
| | | and metabolites | | |
| | | study of nobiletin | | |
| 3 | Franca G., | chondrocytes | National | Natural |
| | Panico A. M., | | Product | Product |
| | Bonina F., | | Research | Research |
| | Messina R., | | | 2010 24:15 |
| | Rizza L., | | | (1469-1480) |
| | Musumeci G., | | | |
| | Rapisarda P., | | | |
| | Cardile V. | | | |
| 4 | Omodamiro O. D., | | Journal of | Journal of |
| | Umekwe C. J. | | Chemical & | Chemical |
| | | | Pharmaceutical | and |
| | | | Research | Pharmaceutical |
| | | | | Research |
| | | | | 2013 5:5 (56-66) |

We claim:

1. An effective and temporary relief of the symptoms and effects of arthritis consisting of a mixture of Orange Oil (Citrus Sinensis) and Peppermint Oil (Mentha Piperita) along with any other vegetable based Oil

2. The arthritic relief mixture of claim 1 wherein the concentration of Orange Oil is between 25%-45% of volume

3. The arthritic relief mixture of claim 1 wherein the concentration of Peppermint Oil is between 10%-20% of volume

4. The arthritic relief mixture of claim 1 wherein the concentration of any other vegetable oil is between 40%-70% of claim 1

5. The method of claim 1 where the administration is topical

6. The method of claim 1 where the administration is injected

7. The method of claim 1 where the application is on a human being

8. The method of claim 1 where the application is on an animal

9. The method of claim 1 where the use is only Orange Oil

10. The method of claims 1 and 9 where Orange Oil is mixed with Peppermint Oil

11. The method of claim 1 where an extract from the following vegetable oil is used, such as Coconut oil or otherwise known as cocas nucifera, Olive Oil or otherwise known as Olea Europaea, Canola Oil, Corn Oil, Cotton seed Oil, Flaxseed Oil, Linseed Oil, Palm Oil, Peanut Oil, Safflower Oil, Soybean Oil or Sunflower Oil.
